(19) United States
(12) Patent Application Publication
(10) Pub. No.: US 2018/0071138 A1
(43) Pub. Date: Mar. 15, 2018

(54) THERMO ELECTRIC COOLING SYSTEMS FOR HYPOTHERMIA THERAPY TREATMENT

(71) Applicants: Wayne T. Armstrong, Placitas, NM (US); Robert D. Battis, Skilman, NJ (US)

(72) Inventors: Wayne T. Armstrong, Placitas, NM (US); Robert D. Battis, Skilman, NJ (US)

(73) Assignee: Extreme Laser Technologies, Inc., Princeton, NJ (US)

(21) Appl. No.: 15/703,971

(22) Filed: Sep. 13, 2017

Related U.S. Application Data
(60) Provisional application No. 62/394,661, filed on Sep. 14, 2016.

(51) Int. Cl. A61F 7/00; A61F 7/02

(52) U.S. Cl. CPC A61F 7/007; A61F 7/02; A61F 2007/0078; A61F 2007/0075; A61F 2007/0008; A61F 2007/0007; A61F 2007/0056

(57) ABSTRACT

Provided are active Thermoelectric Cooling (TEC) apparatuses for the purpose of providing emergency hypothermia therapy treatment to patients.

### CROSS REFERENCE TO RELATED APPLICATIONS

This application is related to and claims priority from U.S. Provisional Patent Application No. 62/394,661 filed on Sep. 14, 2016, by Robert D. Battis, et al. titled “Thermo Electric Cooling Systems for Hypothermia Treatment”, which is incorporated herein by reference in its entirety.

### FIELD OF THE INVENTION

This invention relates to portable Thermo Electric Cooling (TEC) systems for cooling a person's head and selected other body areas to administer Hypothermia Therapy (HT) treatment, and more particularly to administer HT treatment to a patient in an emergency situation.

### BACKGROUND OF THE INVENTION

Hypothermia therapy (HT) is a treatment by which the brain and body core temperature is lowered to 32 to 34 degrees Celsius. When a person goes into cardiac arrest, the heart stops pumping which causes blood to stop circulating to the brain and this can lead to neurological damage. HT appears to slow the rate of damage and protect the brain from further negative effects when blood begins recirculation. Analysis of multiple studies has shown provides critical benefits for people who suffer cardiac arrest, though it remains under-utilized by emergency medical service crews. HT has been included in the American Heart Association's guidelines for cardiac arrest care for nearly a decade.

Typically, patients who suffer cardiac arrest and undergo CPR have two minutes of no blood supply to the brain, according to Dr. Ron Waksman, professor of medicine and cardiology at Georgetown University and associate chair of cardiology at the Washington Hospital Center in WDC. Even if their heart is restored to normal function, they may suffer from brain damage or memory loss. Appling HT immediately by paramedics can reduce the risk of brain damage or memory loss.

Given the body of evidence of the beneficial effects of HT, it is presumed that the main reason HT is under utilized by emergency medical service personnel or paramedics is both the absence of readily available ice packs which is a poor choice for this treatment due to uncontrolled temperatures and the lag time it takes for refrigeration cooling pads or blankets to cool down a patient. Hence the lack of proper field portable and emergency HT devices necessitates the need for Thermo Electric Cooling apparatus. This invention describes both field portable and emergency Thermo Electric Cooling (TEC) systems to remedy this situation and aid the survival of patients with reduced risk of brain damage or memory loss.

### SUMMARY OF THE INVENTION

An aspect of the present invention provides for cooling a person's head and selected other body parts by active cooling using a Thermo Electric Cooling (TEC) apparatus. This apparatus provides instant cooling and unlike ice packs provides temperature control and temperature profiling to within a degree. In addition, this apparatus is available for use 24-7 without special storage or preparation steps unlike ice which must be kept frozen.

In various aspects of the present invention, the TEC apparatus may take on several forms and physical implementations that result in an array of TEC chips being in contact with the body part through a suitable thermally conductive membrane (herein referred to as “contact” TEC).

In another aspect of the invention, first aid or emergency Hypothermia Therapy (HT) treatment is administered to a subject person's head and body parts using a Thermal Electric Cooling (TEC) apparatus. The TEC apparatus includes TEC chips in contact with the head and body parts through a suitable thermally conductive head cap and pads, suitable TEC heat dissipater secured to the TEC chips, suitable handling convenience screens over the heat dissipaters, a suitable battery, a suitable field use portable package and suitable temperature control electronics to maintain TEC temperatures in a closed loop by controlling TEC drive currents.

In another aspect of the invention, the temperature is profiled in accordance with medical protocols.

In another aspect of the invention provides for administering first aid HT treatment to head and body parts by TEC as above but with a closed cycle cooled fluid replacing the heat dissipaters.

In another aspect of the invention administering first aid HT treatment as in described above but with TEC eliminated in the cap and pads thereby cooling the body parts using a fluid alone. In this case the fluid would be cooled remotely from the cap and pads by an external TEC apparatus.

Another aspect of the invention provides for non first aid or emergency treatment where heat is applied to body parts by reversing current in the TEC chips.

Another aspect of the invention provides for cooling or heating either or both the head and selected other body parts.

In another aspect of the invention the apparatus can be used for treatment to cool down a child or adults fever in a controlled fashion without submersing one in an ice bath which shocks a person and can be very uncomfortable and dangerous.

### BRIEF DESCRIPTION OF THE DRAWINGS

FIG. 1 illustrates an exemplary HT Contact TEC System that is useful for understanding the present invention.

FIG. 2 illustrates an exemplary thermally conductive head cap that is useful for understanding the present invention.

FIG. 3 illustrates an exemplary thermally conductive pad that is useful for understanding the present invention.

FIG. 4 illustrates an exemplary prior art single stage conventional Thermo Electric Cooling chip, also known as a Peltier element.

FIG. 5 illustrates an exemplary HT Contact TEC System Variant that is useful for understanding the present invention.

FIG. 6 illustrates an exemplary HT Contact TEC Remote Cooling System that is useful for understanding the present invention.

FIG. 7 illustrates two examples of state of the art thermally conductive flexible substrates that are useful for understanding the present invention.

### DETAILED DESCRIPTION

In the following description, for purposes of explanation, specific numbers, materials and configurations are set forth in order to provide a thorough understanding of the invention. It will be apparent, however, to one having ordinary skill in the art, that the invention may be practiced without these specific details. In some instances, well-known features may be omitted or simplified so as not to obscure the present invention. Furthermore, reference in the specification to “one embodiment” or “an embodiment” means that a particular feature, structure or characteristic described in connection with the embodiment is included in at least one embodiment of the invention. The appearances of the phrase “in an embodiment” in various places in the specification are not necessarily all referring to the same embodiment.

FIG. 4 illustrates a prior art single stage conventional Thermo-Electric Cooling (hereinafter, “TEC”) chip 100, also known as a Peltier element. TEC operates according to the Peltier effect. The effect creates a temperature difference by transferring heat between two electrical junctions. A voltage is applied across joined conductors to create an electric current. When the current flows through the junctions of the two conductors, heat is removed at one junction, Cold Side 120, and cooling occurs, while heat is transferred to the opposite junction, Hot Side 140. Many TEC chips are combined to form a TEC assembly and an assembly may be combined with other items, such as thermal conducting sheet or blanket, heat dissipater and battery to form a complete system. It is also noteworthy that reversing the direction of current flow in the TEC Chip 100 effectively acts to reverse the direction of heat transfer, so that the cooling apparatus described herein may alternatively operate as a heating apparatus, in many cases.

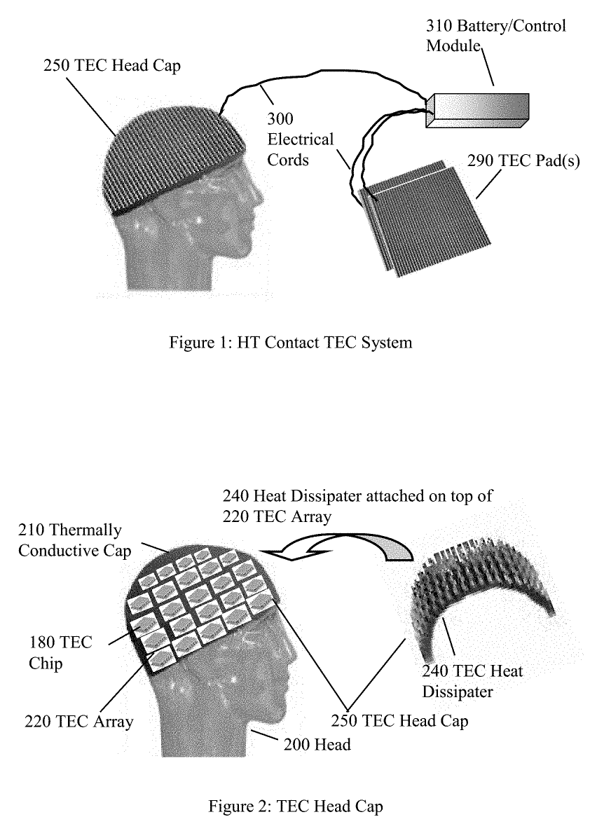

An embodiment of the present invention, called the HT Contact TEC System illustrated in FIG. 1, advantageously provides for instantly cooling head and body parts by direct contact of TEC imbedded in a suitably designed TEC Head Cap 250 and TEC Pad 290. The TEC Pad(s) may be optional.

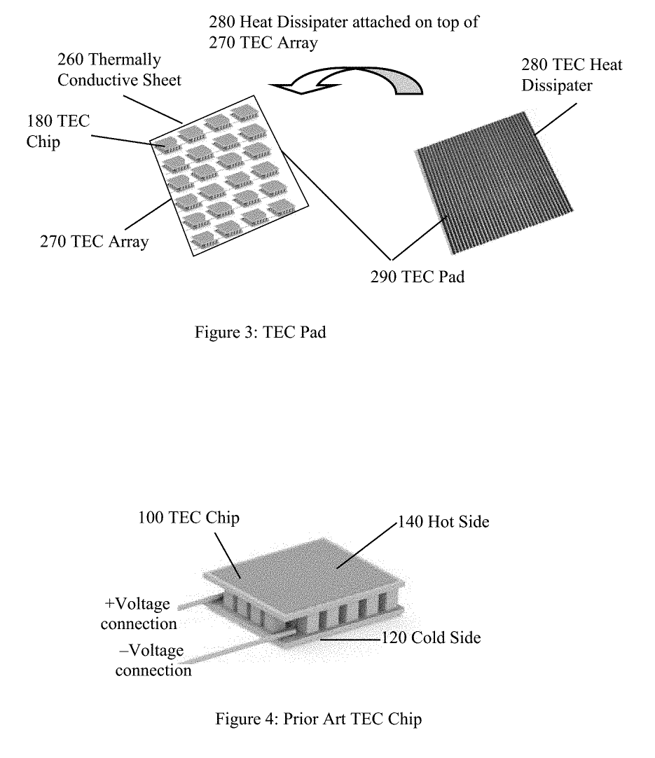

Each cooling element, such as the TEC Head Cap 250 of FIG. 2 and the TEC Pad(s) 290 of FIG. 3, use arrays of TEC Chip 100 such as in FIG. 4, with each chip providing instant cooling due to its low mass and Peltier physics as the current is applied to the chip.

One TEC chip suitable for use in exemplary embodiments of the present invention is the TE-127-1.0-1.5, available from TE Technology, Inc., 1590 Keane Drive, Travis City, Mich. 49696, http://tetech.com/.peltier-thermoelectric-cooler-modules/. Many TEC chips may be combined to form a TEC assembly and an assembly may be combined with other items, such as thermal conducting blankets or pads, fluid heat transfer unit, battery, electronics and housing to form a complete apparatus.

The TEC Head Cap 250 (FIG. 2) consists of a Thermally Conductive Cap 210 with an attached TEC Array 220. This array consists of TEC Chips 100 (FIG. 4). Attached to the TEC 140 Hot Sides in the TEC Array 220 is a suitably designed TEC Heat Dissipater 240. This heat dissipater or radiator may take on various forms to include but not limited to segmented articulated aluminum fins and micro fans. FIG. 2 illustrates an exemplary thermally conductive head cap with an attached array of TEC chips connected to the cap on the TEC cold sides. The TEC hot sides are attached to an outer layer of suitably designed fins or equivalent as a heat dissipater.

FIG. 3 illustrates an exemplary thermally conductive pad with an attached array of TEC chips thermally connected on the TEC Cold Sides 120. The TEC hot sides are attached to a top layer of suitably designed fins or equivalent as a heat dissipater. Each TEC Pad 290 consists of a Thermally Conductive Sheet 260 with attached TEC Array 270. This array consists of TEC chips 100. Attached to the TEC Hot Sides 140 in the TEC Array 270 is a suitably designed TEC Heat Dissipater 280. This heat dissipater or radiator may take on various forms, including, but not limited to, segmented articulated aluminum fins and micro fans. The TEC Cool Pads 290 may be thought of as a flexible flat version of the head contoured TEC Head Cap 250, with or without some interchangeable parts. The TEC pad 290 may be any reasonable size and shape to fit various parts of the body.

FIG. 1 illustrates an embodiment of the HT Contact TEC System consisting of a FIG. 2 TEC Head Cap 250 and FIG. 3 TEC Pads 290, plus a Battery/Control Module 310 and Electrical Cords 300 connecting the elements. Not illustrated is a suitable portable carry case. In this example the HT Contact TEC System combines one TEC Head Cap 250 and two TEC Cool Pads 290, but other combinations are possible. The Battery/Control Module 310 controls the temperature of all cooling elements, e.g., TEC Head Cap 250 and TEC Cool Pad(s) 290, to within a degree of the desired temperature using an imbedded thermistor or equivalent temperature monitoring device in each of the cooling elements. Each cooling element is cooled to a preset temperature by controlling the current through the corresponding group of TEC units. As a result of electronic temperature control each cooling element may be programmed to fit a pre-established medical protocol, for example: the temperature can be lowered initially then raised as a function of time only, body part temperature and ambient temperature measured in real time. Another option is to provide different cooling temperatures at different locations within the TEC Head Cap 250, for example: the forehead temperature may be cooled more than the scalp and this temperature gradient profile may be changed to fit a pre-established medical protocol.

In an embodiment of the HT Contact TEC System, the Battery/Control Module 310 (FIG. 1) will control all element temperatures using a programmed small microprocessor and interface electronics. The microprocessor will accept inputs from various temperature monitoring devices to include not only TEC cold sides but TEC hot side temperatures, patient skin temperature and ambient temperature. The microprocessor through interface electronics will appropriately control TEC drive current to each set of TEC units to implement the programmed HT temperature treatment protocol(s), or other treatment protocols. Appropriate controls may be provided to implement these protocols. These controls may take the form of switches, push buttons, a menu driven alphanumeric display or a combination of these. In addition and because TEC may provide either cooling or heating depending on the direction of current through the TEC units a separate cooling/heating control may be provided. TEC cooling (HT treatment) should be the baseline with heating as the alternate or option. This separate control may therefore be a heating control and for patient safety the control may be connected to an LED warning light and or piezoelectric audible alarm to implement TEC element heating of the TEC Head Cap 250 or TEC Pad(s) 290 in contact with the patient's body.

As a result of the heat dissipated by the Heat Dissipater 240 and Heat Dissipater(s) 280 the corresponding elements will be hot to the touch during removal of the elements from the body. If this elevated temperature turns out to be uncomfortable a convenience screen may be installed over the heat dissipaters so emergency personnel may avoid touching the heat dissipaters. These convenience screens are not illustrated in FIGS. 1, 2 and 3 in order to simplify these figures.

One embodiment of the HT Contact TEC System, as depicted in FIG. 1, is completed by adding a suitable battery and carrying case. The Battery/Control Module 310 may contain a single-use or rechargeable battery with the battery having a quick replacement feature. For portable use in the field such as an ambulance it should have a cigarette lighter type, or similar charging cord. A standard electrical cord may alternatively be employed for non-mobile use.

In one embodiment of the carry case for the HT Contact TEC System an appropriate means should be provided to attach or hang the case from a portable patient transport unit such as a stretcher.

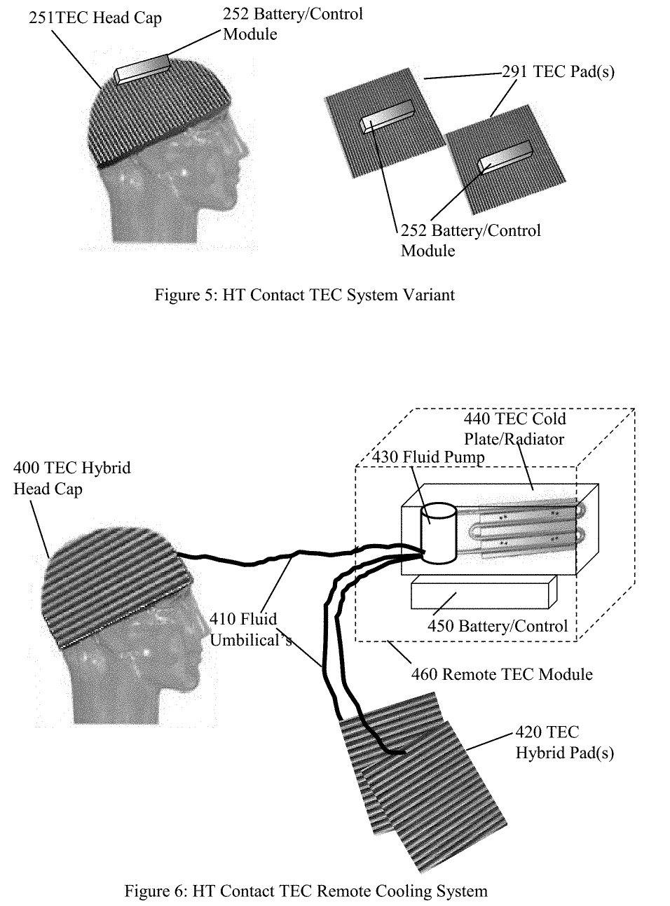

In another embodiment of the HT Contact TEC System titled HT Contact TEC System Variant, such as depicted in FIG. 5, each element (cap and pad(s)) in contact with the patient's body has its separate Battery/Control Module 252. These separate Battery/Control Modules may be the same or different modules. The TEC Head Cap 251 and TEC Pad 291 have the same construction as there corresponding TEC Head Cap 250 and TEC Pad 290 except for the direct attachment of the battery and control modules.

Each attached Battery/Control Module 252 may contain the same battery or batteries, or the battery may be sized to accommodate different amp-hour requirements of the element in contact with the patient's body.

Each attached Battery/Control Module 252 may contain the same control unit with each control unit containing a small microprocessor and interface electronics to control the temperature and temperature profiling of the TEC within the TEC Head Cap 251 and TEC Pad(s) 291. The design of this control unit will be similar to the design of the control unit in the Battery/Control Module 310 in terms of temperature monitoring, functional control of modes including cooling and heating and protocol programming. The design of this control unit will be simpler than the control unit in the Battery/Control Module 310 since it only controls one set of TEC units in its own element and only has one set of temperature monitoring units as inputs to the microprocessor.

In another embodiment of the HT Contact TEC System Variant of FIG. 5 the Battery/Control Module 252 may be split into two parts with each TEC Head Cap 251 and TEC Pad 291 having its own control module and with a common battery separate and remote.

In embodiments of the HT Contact TEC System and HT Contact TEC System Variants benefits include simplicity, field portability, flexibility in applying elements to body areas, immediate and rapid cooling, programmed temperature control and ease of use through thoughtful layout of controls and labeling, guided by accepted medical protocols. In comparing the HT Contact TEC System with the HT Contact TEC System Variants a tradeoff is made to eliminate the wiring, single battery and control of the HT Contact TEC System in favor of self contained but more complex TEC Head Cap 251 and TEC Pad(s) 291 of the HT Contact TEC System Variants.

Another embodiment of the present invention called HT Contact TEC Remote Cooling System advantageously provides for removal of heat from the TEC Head Cap 250 and TEC Pad(s) 290 (FIG. 1) using a Remote TEC Module 460 (FIG. 6) comprising a closed cycle cooled fluid from a TEC Cold Plate/Radiator 440 acting through a Fluid Pump 430 and Fluid Umbilical's 410. This cooled fluid circulates through TEC Hybrid Head Cap 400 and TEC Hybrid Pad(s) 420.

In this HT Contact TEC Remote Cooling System (FIG. 6) the design of the TEC Heat Dissipater 240 (FIG. 2) and Heat Dissipater 280 (FIG. 3) are modified to include a leak proof fluid chamber in which a closed cycle fluid may circulate. This cooling fluid controls/removes the heat from all Hot Side 140 of the TEC Chips 100, transferring this heat to the remote TEC Cold Plate/Radiator 440. The modified TEC Heat Dissipater 240 and Heat Dissipater 280 both with circulating fluid channels are herein referred to TEC Hybrid Head Cap 400 and TEC Hybrid Pad(s) 420, respectively.

The TEC Cold Plate/Radiator 440 (FIG. 6) may contain/incorporate any appropriate prior art cold plate, water block or serpentine tube attached to an appropriate heat dissipater/fan module.

In this HT Contact TEC Remote Cooling System as shown in FIG. 6, cooling to the body parts is controlled by controlling element TEC drive current in the same manner as described for the HT Contact TEC System. Adequate heat removal from the arrays of TEC units in each cap and pad element is controlled by the Battery/Control unit 450 by closed cycle control of fluid temperature. The fluid temperatures are monitored using thermistors or similar temperature monitoring devices imbedded in the return fluid hoses within the Fluid Umbilical's 410. In this design fluid temperatures need not be controlled precisely but controlled to avoid thermal runaway and failure of the TEC Chips 100. The TEC Cold Plate/Radiator 440 is designed to provide a reasonably cool fluid temperature in order to increase the TEC Chips 100 operating efficiency. As the TEC Chips 100 operating efficiency increases, the battery capacity/size may be reduced without sacrificing operating time.

Modifying the TEC Heat Dissipater 240 (FIG. 2) and Heat Dissipater 280 (FIG. 3) to accommodate closed cycle cooled fluid as in the HT Contact TEC Remote Cooling System of FIG. 6 basically trades off heat removal from the elements in the HT Contact TEC System with the added complication of Fluid Umbilical's 410, Fluid Pump 430 and TEC Cold Plate/Radiator 440. Removing this heat from the HT Contact TEC System means the elements will be cool to the touch. All other benefits of the HT Contact TEC System remain such as field portability, flexibility in applying elements to body areas, immediate and rapid cooling, programmed temperature control and ease of use through thoughtful layout of controls and labeling, guided by accepted medical protocols.

One embodiment of the HT Contact TEC Remote Cooling System as depicted in FIG. 6 is completed by adding a suitable battery and carrying case. The Battery/Control Module 450 may contain a single-use or rechargeable battery with the battery having a quick replacement feature. For portable use in the field such as an ambulance it should have a cigarette lighter type, or similar charging cord.

Another embodiment of the HT Contact TEC Remote Cooling System of FIG. 6 is to eliminate the TEC Chips 100 from the TEC Head Cap 250 and TEC Pad(s) 290 and allow the closed-cycle cooled fluid circulating through the modified TEC Heat Dissipater 240 and TEC Heat Dissipater 280 to cool the body parts. This embodiment of the remote cooling concept has the advantage of simplifying the design of TEC Head Cap 250 and TEC Pad(s) 290 but has the extreme disadvantage of not allowing instant cooling afforded by the Contact TEC approach. Not allowing instant cooling for HT can of course be mitigated by pre-cooling the closed-cycle fluid in the remote cooling system but this complicates the 24-7 availability afforded by the HT Contact TEC System of FIG. 1. In addition, programmed temperature profiling with the cooled fluid alone is not possible as it is with the direct contact TEC Systems. Poor temperature control and poor temperature profiling of this embodiment wherein the TEC units are eliminated from the TEC Head Cap 250 and TEC Pad(s) 290 also applies to hypothermia treatment when the fluid is heated.

In one embodiment of the carry case for the HT Contact TEC Remote Cooling System an appropriate means is provided to attach or hang the case from a portable patient transport unit such as a stretcher or gurney.

For all embodiments of the present invention the HT Contact TEC System, HT Contact TEC System Variants and HT Contact TEC Remote Cooling System the Thermally Conductive Cap 210 (FIG. 2) and Thermally Conductive Sheet 260 (FIG. 3) must exhibit three attributes: 1) High degree of thermal conductivity, 2) Flexibility and 3) Structural integrity. A high degree of thermal conductivity is required in order to efficiently transfer the heat from a person's skin to the cold sides 120 of the TEC chips. Flexibility of both the cap and Blanket is required in order to maximize the surface contact between the cap and sheet and the person's skin, or in the case of body hair to compress the body hair so as to remove as much air as possible. Maximizing the surface contact has an additional benefit by trapping skin moisture under the conductive surface thereby enhancing heat transfer from the body to the TEC units. Structural integrity is required to build-in high reliability to counter the abuse prevalent in field portable service devices.

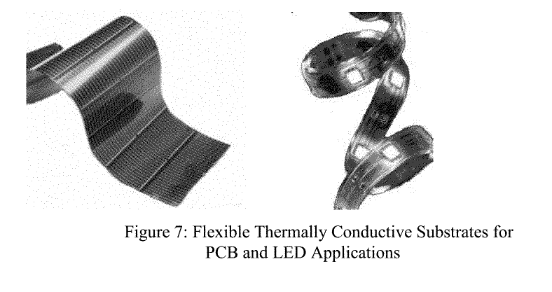

The thermally conductive materials of both the Thermally Conductive Cap 210 and Thermally Conductive Sheet 260 may be composites made up of, but not limited to, thin copper/aluminum/silver bands or wire, woven to form a cloth with other materials such as glass, conductive flexible epoxy and aluminum nitride. The conductive materials may comprise high thermal crystallite filled molecularly flexible polymers or other state of the art materials being developed for the high thermally conductive flexible printed circuit board (PCB) and Light Emitting Diode (LED) industry. FIG. 7 illustrates two examples of state of the art thermally conductive flexible substrates for PCBs and LEDs.

Engineering the Thermally Conductive Cap 210 and Thermally Conductive Sheet 260 can take advantage of the advances in thermally conductive flexible substrates for the printed circuit board and LED industries. One material usable as a thermally conductive flexible substrate is COOL-CLAD™ available from AVANTE International Technology, Inc., http://www.aitechnology.con/products/insulated-metal-substrates/.

In an embodiment of the present invention all the HT Contact TEC System, HT Contact TEC System Variants and HT Contact TEC Remote Cooling System use TEC chips 100, but these chips may be different in size and power but in all cases it is anticipated that TEC will be done using a single stack TEC Chip 100 as illustrated in FIG. 4.

In any embodiment of the present invention the batteries indicated for the HT Contact TEC System, HT Contact TEC System Variants and HT Contact TEC Remote Cooling System may be any suitable type, single use or rechargeable and if rechargeable may use any appropriate recharging method. It is anticipated that the preferred battery will be rechargeable Lithium ion due to their superior current capacity to weight ratio.

In an embodiment of the present inventions the HT Contact TEC System, HT Contact TEC System Variants and HT Contact TEC Remote Cooling System are suitable for use as a temporary portable first aid device to administer HT treatment to lower selected body part temperatures to critically ill patients as well as to other non critical patients. TEC cooling is better than cold packs or ice packs because the target temperature administered to the patient can be electronically regulated and profiled to within a degree if desired. And in addition the HT treatment can be available anywhere at any time (available 24-7) by just turning on the TEC system. In addition, these systems can provide hyperthermia treatments to immediately raise selected body part temperatures in accordance with medical protocols in patients subjected to frost bite or suffering from hypothermia.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A Thermal Electric Cooling (TEC) apparatus comprising:
a plurality of TEC chips in thermally conductive communication with a hot radiator on a hot side of the TEC chips and a thermally conductive head cover, head wrap, head band, curved pad or flat pad (body cover) on a cold side of the TEC chips, with the hot radiator positioned on the exterior side of the body cover and the TEC chips cool sides thermally attached to the Body Cover, the TEC chips further attached to a controller unit attached to a battery, the controller unit comprising a variable temperature setting for modulating the current flow from the battery to the TEC chips.

2. The TEC apparatus of claim 1, wherein the hot radiator further comprises a fan connected to the controller and battery for assisting the removal of hot air.

3. The TEC apparatus of claim 1, wherein the controller and battery are co-located on the body cover

4. The TEC apparatus of claim 1, wherein the controller and battery are separate from the body cover.

5. The TEC apparatus claim 1, wherein the body cover set point temperature varies in temperature across the body cover area.

6. The TEC apparatus claim 1, wherein the body cover set point temperature varies over time.

7. The TEC apparatus of claim 1, wherein the hot radiator is immersed in a closed cycle thermally conductive cooled fluid for the removal of heat from the TEC chips hot side.

8. The TEC apparatus of claim 1, further comprising:
a thermocouple attached to the body cover in communication with the controller and battery, wherein the controller further comprises a variable temperature setting and, in operation, current to the TEC chips is modulated to maintain the body cover temperature setting.

9. The TEC apparatus of claim 1, further comprising:
one or more thermally conductive body cover(s) with leak proof fluid channels wherein closed cycle thermally conductive cooled fluid is circulated for the purpose of cooling the body covers which in turn cool the body part(s), with the cooled fluid being cooled remotely by a TEC apparatus comprising a thermocouple, controller, fluid pump, fluid cold plate, TEC hot side radiator and battery, wherein the controller further comprises a variable temperature setting and, in operation, current to the TEC chips is modulated to maintain the fluid temperature which in turn controls the desired body cover temperature.

10. The TEC apparatus of claim 1, wherein heat is applied to body parts by reversing current in the TEC chips.
